(19) United States
(12) Patent Application Publication
(10) Pub. No.: US 2009/0181923 A1
(43) Pub. Date: Jul. 16, 2009

(54) Method of increasing weight gain and reducing diarrhea morbidity, mortality and severity by stimulation of natural immune response, nutritional support of immune function and supplemental nutricines and probiotics

(76) Inventor: Robert Terrell Winn, Big Sandy, TX (US)

Correspondence Address: Robert T. Winn DVM, 141 PR 3808, Big Sandy, TX, 75755, US

(21) Appl. No.: 12/217,554

(22) Filed: Jul. 8, 2008

Related U.S. Application Data
(62) Division of application No. 10/923,313, filed on Aug. 23, 2004, now abandoned.

(51) Int. Cl. A61K 31/715

(52) U.S. Cl. 514/54

(57) ABSTRACT

A method for the promotion of growth and weight gain, the abatement of diarrheal disease and the reduction in mortality in a farm animal comprising administering bacterial polysaccharides derived from dried rumen fluid combined with nutritional aids is described. The use of a specialized nutritional composition the first few days of young animals' lives results in decreased diarrhea morbidity, severity and mortality. It also helps supply nutrients for the support of natural immune response and function.

### CROSS-REFERENCES

This application is a Divisional application of application Ser. No. 10/923,313, filed on Aug. 23, 2004, entitled “Animal Nutritional Product that Increases Weight Gain and Reduces Diarrhea Morbidity, Mortality and Severity by Stimulation of Natural Immune Response, Nutritional Support of Immune Function and Supplemental Nutricines and Probiotics.”

### FEDERALLY SPONSORED RESEARCH OR DEVELOPMENT

The development and research for this invention involved no federal or state funding. It was supported in full by private funding.

### COMPACT DISCS AND ELECTRONIC DATA

There are no electronic data or compact discs included with this submission.

### DETAILED DESCRIPTION AND SPECIFICATION

### Field of the Invention

The present invention relates to the collection, processing, and sterilization. It further relates to the subsequent utilization of this product with a mixture of probiotics, nutricines, vitamins, minerals, an amino acid, and a monosaccharide. In particular this invention involves feeding this mix to young animals for the first few days of life to increase weight gain, reduce diarrhea severity, morbidity and mortality by stimulation and support of the animals natural immune response.

### BACKGROUND OF THE INVENTION

Animals are raised in concentrated rearing units. These units are used on a constant basis resulting in a build up of contamination and disease organisms. The young newborn animals are frequently affected with diarrhea. Although management practices to maximize the passive immunity are used and sanitation measures followed to minimize the exposure of newborns to virulent organisms, the diarrheal disease process is the most costly disease process affecting the rearing of newborns.

There is both a political move and a public health concern with the use of antibiotics as feed additives. There are also public health concerns with the extra-label use of antibiotics in food producing animals. To maintain health and increase productivity without the use of antibiotics is the goal of many endeavors at this time (Donovan, D. C., et al, Growth and Health of Holstein Calves Fed Milk Replacers Supplemented with Antibiotics or Enteroguard, 2002, J. Dairy Sci. 85:947-950: Webb, P. R., et al, Addition of fructooligosaccharide (FOS) and sodium diacetate (SD) plus decoquinate (D) to milk replacer and starter grain fed to Holstein calves, 1992, J. Dairy Sci. Vol 75 Suppl. 1:300). As such, there are many studies and products, which attempt to increase the immuno-competence of the neonate. Vaccines, serum immunoglobulins, colostrum replacers and colostrum antibody preparations have all been used to improve the neonate's immune status. Other nutritional supplements have been described. U.S. Pat. No. 6,667,063 B2 describes a composition containing as the essential ingredients colostrum, a selected whey product and defined amounts of selenium or an organic or inorganic, water soluble selenium precursor. The goal, ingredients and method of action are different from the present composition.

Most of the ingredients of this composition product are currently commercially available. The combination is not currently commercially available and the resulting increase in weight gain, lowered number of treated animals and increased livability of the animals is surprisingly better than expected from the individual products alone. This type of response has been seen in ruminating animals as taught in U.S. Pat. No. 4,405,609. Most feed additives have to be fed for the full or at least an extended feeding period to achieve results. This composition product is only fed for the first 3-7 days of life and achieves results. Another composition is described in U.S. Pat. No. 6,258,399, which improves the immune system indirectly by providing early carbohydrate digestion. This increase in energy stimulates the growth stimulation hormones, which in turn starts the growth and response of the immune response system. Similarly, it is only fed for the first days of life with continued results and then the chicks are switched to the usual feed. The composition of the current patent contains bacterial polysaccharides, vitamins, organic minerals and probiotics, has a different effect on the animal by causing a localized immune response, and does not supply a disaccharide or an oligosaccharide as described in the composition of U.S. Pat. No. 6,258,399.

Another composition is described in U.S. Pat. No. 6,365,152 B1. This composition is used for the treatment of scours and not the prevention of scours. It contains many of the same nutrients found in the current patent. However, it claims the mix is a solution of trace mineral, which is mixed with other ingredients including kelp. We are taught that kelp is a natural source of carbohydrates, amino acid, vitamins, minerals and trace elements. We are further taught that kelp also contains over 60 minerals and elements including iodine, 21 amino acids, fiber, simple and complex carbohydrates. Kelp is added at the rate of 1 gram per treatment. There is no kelp in my current patent. Also, the current patent contains approximately 5 grams of the purified amino acid threonine per each daily ration for calves of the current composition, compared to the small amount that might be found in one gram of kelp. There are no bacterial polysaccharides included into the composition of U.S. Pat. No. 6,365,152 B1. Also, the referenced patent is for treatment of scours in farm animals, not for the prevention of scours and increased weight gain. A second composition for the treatment of scours contains three energy sources and is described in U.S. Pat. No. 6,066,341.

One of the objectives of this invention is to provide nutrients that increase the ability of the young animal to develop its own immunity. B complex vitamins, and organic trace minerals are added to the formulation to insure that all enzymatic activity may occur without compromise due to a deficiency of catalytic enzymes. Due to the expense of these products, they are not added to milk replacers used in rearing calves. These products do not occur in whole milk from the dam of mammals in sufficient levels without supplementation. Due to the low dry feed intake the first few days of life, it is unlikely that young mammals eat sufficient feed to obtain the recommended level of intake of these nutrients for optimum production.

These vitamins and minerals may be added to diets of young chicks but due to the inanition or lowered dry feed intake the first few days of life, it is unlikely that sufficient feed is eaten to obtain the recommended level of intake for optimum production. As taught in U.S. Pat. No. 6,733,759 B2, a specialized method of feeding the newborn chicks must be used. The ingredients used in this product may be administered to poultry by using the unique delivery system described in the aforementioned patent or a similarly devised delivery system.

A second objective of this invention is to provide specially prepared and selected probiotics that can be used to help keep the flora of the gut populated with bacteria that support health of the gut and over grow or suppress the virulent bacteria. Methods of collection and culture of these specially derived probiotics are described in U.S. Pat. Nos. 4,689,226; 6,214,335 B1; and 6,645,530 B1. Continuously fed probiotics have been shown to improve body weight gain, feed conversion, and fecal condition of newborns (Abe, F., N. Ishibashi and S. Shimamura, Effect of Administration of Bifidobacteria and Lactic Acid Bacteria to Newborn Calves and Piglets, 1995, J. Dairy Sci. 78:2838-2846). These products are available as dry feed inclusions and also as individual innoculants to be administered orally on a daily basis as described in U.S. Pat. Nos. 4,985,246; 5,718,894 and 5,902,578. Probiotics are added to other treatment packages currently available, but are not commonly added to powdered milk replacers nor are seldom used as additives in whole milk fed to calves. They may be added to diets of young chicks or pigs, but due to the low dry feed intake the first few days of life, it is unlikely that sufficient feed is eaten to obtain the recommended level of intake for optimum inoculation. Another method of inoculation of probiotics into chicks is by spraying a suspension of viable microorganisms of lactic acid bacteria on newborn chicks within 4 days of hatching, U.S. Pat. No. 6,410,016 B2. By adding them to the composition, a more complete stimulant package is formed, and specific strains known to be advantageous to the specific species of animal being fed may be used. In most situations, only one probiotic mixture/package is available in a feed mill, and this is used in the feed produced regardless of the species for which the feed is intended.

U.S. Pat. No. 5,374,425 describes the manufacture of a killed probiotic. The stabilization process is somewhat similar to the process used in the current invention. Both products are autoclaved to kill the bacterial cells. In the current invention, autoclaving takes place at 116° C. for 45-60 minutes at a pressure of 10 p.s.i. The referenced patent uses a variable temperature (100° to 121° C.) and a shorter duration (15-30 minutes). Also there is also a difference in drying. To separate the bacteria cells in U.S. Pat. No. 5,374,425 a flocculating agent is added to the culture and the cells are allowed to settle out. The liquid is decanted off. Heat, spray or freeze-drying is promoted as acceptable drying methods and the use of a drying agent is proposed. These methods except for freeze-drying are not acceptable in the current invention. Another difference is that the current patent uses rumen fluid bacteria, while this patent uses a specific culture or mixes of dried specific cultures. U.S. Pat. No. 4,021,303 also produces killed organisms. This process includes chemically treating the microorganisms with alkali at a pH of 10.5-12.9 and a temperature of 0°-30° C., washing with water and mechanically rupturing the bacteria at a pH of 7-10.2.

Another aim of this invention is to increase the natural local immune response by the exposure of the gut to bacterial polysaccharides in a measured, safe and controlled manner. Rumen fluid has been shown to increase growth rate in calves, decrease morbidity, mortality and use of treatments for diarrheal disease (Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656). Rumen fluid has been shown to contain bacterial polysaccharides. These bacterial polysaccharides are considered the “active ingredient” in rumen fluid. Bacterial polysaccharides have been shown to elicit localized immunity. Rumen bacteria have been reported to have extracellular polysaccharide “coats” that are similar to those found on many Gram (−) organisms (Costerton, J. W., H. N. Damgaard and J. K. Cheng, Cell envelope morphology of rumen bacteria, 1974, J. of Bacteriology, 118:1132-1143). It is my belief that this similarity is the reason ruminal fluid bacteria are the best to use for this desired result.

We are taught in U.S. Pat. No. 6,444,210 B1 that bacterial polysaccharides have been used as vaccines to enhance specific humoral immunity and in the particular invention named they are used to enhance general cellular immunity against a wide variety of microorganisms. The mentioned patent describes a method of isolation, purification, stabilizing and using Brucella abortus and Yersinia enterocolitica outer polysaccharide as an immunizing agent. This differs from the current invention in that the current invention makes no strides toward selecting, isolating or purifying a particular polysaccharide considered effective as an immune modulator. It further differs from the current invention in that the current invention makes no effort toward selecting, isolating or purifying the bacterial polysaccharide from the rest of the ingredients in the rumen fluid, except for excluding physically large fibers and particles. Also, the number of species of bacteria in the rumen is great and there are no steps taken to reduce this number of species. Three other similar claims have been made for specific extracts of polysaccharides to be used as vaccinal agents, see U.S. Pat. Nos. 4,210,641; 6,007,818; and 6,045,805. The current invention differs from these three inventions for the aforementioned reasons.

We are told in U.S. Pat. No. 6,087,342 that the extraction of polysaccharides that have immune stimulating properties results in small fragments of the longer chain immune-stimulating polysaccharides. These fragments that occur have lower bioactivity than that found in the parent substance. This patent involves the use of a special substrate to bind the small fragments to which potentates the activity of the fragments. This differs from the current invention in two main aspects. First an isolated product in the form of bacterial polysaccharides or bacterial nucleic acids from bacteria is used. Second this is bound to a specialized substrate. My invention uses the whole rumen fluid, or the whole bacterial culture, as it were. I also use the rumen ingesta smaller than 2 mm as the substrate that is used to carry the bacteria.

Another novel method of stimulating the immune system with bacterial produced products is described in U.S. Pat. No. 5,840,318. This method consists of growing bacteria in a stressed manner to increase the stress response factors production of the bacteria. These products are then isolated and used to activate and modulate circulating macrophages. This differs from the current invention in several methods, but primarily due to the fact that the bacteria are stressed instead of grown to peak growth rates. The stress response factors desired by the described method are not a consideration in the current invention.

Bacterial polysaccharides are produced under several patents for use as food thickeners. These patents use bacteria of the genus Xanthomonas and describe a process to grow the bacteria using specialized media or growing conditions. These descriptions are found in U.S. Pat. Nos. 3,328,262; 3,391,061; 3,433,708; and 4,692,408. Other bacterial polysaccharides are produced for use as viscosity regulators used in various manufacturing processes as described in U.S. Pat. No. 4,567,140.

Rumen fluid fed fresh has resulted in increased growth rate in calves, decreased morbidity, mortality and use of treatments for diarrheal disease (Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656). The obvious problems to using fresh rumen fluid are the daily collection of the fluid. The chance of spreading disease. The need to maintain a fistulated animal on each farm. Rumen fluid may be sterilized and bottled to increase storage time. However, upon opening, the bottle must be refrigerated. Also, each farm would need to maintain the equipment to sterilize the rumen fluid.

Another problem is that there is no way to accurately measure the bacterial polysaccharide content of the rumen fluid daily on the farm. It has been shown that the number of rumen bacteria are affected by time of day, diet, time following feeding, location of sampling and diet physical characteristics (Bryant, M. P., and 1. M. Robinson, Effects of Diet, Time After Feeding and Position Sampled on Numbers of Viable Bacteria in the Bovine Rumen, 1968, J. Dairy Sci., 51:1950-1955; Bryant, M. P., and I. M. Robinson, An Improved Nonselective Culture Media for Ruminal Bacteria and its use in Determining Diumal Variation in Numbers of Bacteria in the Rumen, 1961, J. Dairy Sci., 44:1446-1456). The result is a varying level of rumen bacterial polysaccharide content collected. This phenomenon was observed by other workers (Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656).

The process of the current invention allows for the collection of rumen fluid; sterilization of the fluid to prevent disease spread; maximization of the bacterial polysaccharide in the fluid collected by proper timing of feeding and collection, and by specialized ration formulations to increase bacterial growth in the rumen. One of the main objectives of this process is to produce as large a population of ruminal bacteria as possible and harvest them during peak concentration. There are many patents that deal with growing bacteria. The major difference found in the current invention is the use of a cow as an apparatus for the growth of bacteria. Also instead of liquid purified substrates this invention uses standard cow feeds as a substrate to produce the bacteria growth. The cow's rumen is considered to be an anaerobic growing situation. U.S. Pat. Nos. 3,002,894; 4,752,564; and 5,660,977 all deal with aerobic bacterial growth. Several of the patents deal with culturing processes that in some way control the growth of the culture. U.S. Pat. Nos. 4,021,304; 5,017,479; and 6,284,453 are all in this category but no apparatus is claimed in the patent. An apparatus is claimed in U.S. Pat. Nos. 2,686,754; 2,767,118; 3,010,881; 3,018,224; 3,227,557; 3,672,953; 3,766,010; 3,767,534; 3,880,716; 4,167,450; 4,230,806; 4,865,969; 4,900,669; 5,316,905; 5,541,056; and 6,716,617 B1.

U.S. Pat. No. 4,228,275 describes a process of producing a nitrogen containing polysaccharide. This process includes the use of a specific bacterium, not many species of bacteria as is found in the rumen. It also requires reaction with and aqueous ammoniacal solution at a temperature of 100° to 250° C. The resulting product is used to control viruses in plants. U.S. Pat. No. 4,529,701 describes a method of stimulating bacterial growth in an anaerobic digestion system. It specifically deals with improving digestion in sewage digestion systems that have gone sour and uses a mixture of an inhibitory ion regulation component and an inorganic pyrophosphate-containing compound.

This is not the first process to take advantage of products produced by microorganisms. I would draw your attention to some patented processes that may on first glance appear similar to this process. In U.S. Pat. No. 6,255,080 B1 rumen bacteria of the Butyrivibrio spp. are used to produce proteinaceous antibiotics that are resistant to gastric proteases, exhibit a high level of hydrophobicity, and are effective under anaerobic conditions. The Butyrivibrio spp. are isolated and cultured and screened for their production of bacteriocin-like activity. In U.S. Pat. No. 1,818,781 mixed cultures of bacteria were used to cause specialized fermentation to produce ethyl alcohol, lactic acid, butyric acid, butyl alcohol, isopropyl alcohol, acetone, etc. Neither patent uses the same growth media, apparatus nor obtain the same end product as the current application.

An additional intention of this invention is to help the immune system by supplying extra antioxidants for the first few days of life to help build up the body stores. Vitamins A and E in addition to the trace minerals copper, zinc and selenium are all important antioxidants. As an additional source of these nutrients, this product allows for the stores of these nutrients to be built up.

Another objective of this invention is to provide an alternate supply of the amino acid threonine for the first few days of life. This is to insure that the diet is not deficient in the amino acid that is most needed in the formation of mucous in the gut. This is an additional advantage in that this amino acid is not specifically added to dry feeds for young animals nor is if found in high levels in whole milk or milk replacer.

A final goal of this invention is to use only AFFCO approved products.

This product may be administered orally to individual animals by either drenching or dosing with a solution of the product. It may be fed to individual animals by mixing it into the milk fed to that animal. It may be fed by some unique system as as has been described above for poultry. Or, it may be top-dressed on dry feed for swine and poultry. The feeding period will range from 3-7 days and should start on day 1 or 2 of life.

### SUMMARY OF THE INVENTION

A method for the promotion of growth and weight gain, the abatement of diarrheal disease and the reduction in mortality in a farm animal comprising administering bacterial polysaccharides derived from dried rumen fluid combined with nutritional aids.

### DRAWINGS

There are no drawings.

### SPECIFICATION

The invention A Method of Increasing Weight Gain and Reducing Diarrhea Morbidity, Mortality and Severity by Stimulation of Natural Immune Response, Nutritional Support of Immune Function and Supplemental Nutricines and Probiotics is a method.

### Method

The invention A Method of Increasing Weight Gain and Reducing Diarrhea Morbidity, Mortality and Severity by Stimulation of Natural Immune Response, Nutritional Support of Immune Function and Supplemental Nutricines and Probiotics as stated above is actually a method of use of a composition of matter. The process of producing the raw material used in the production of the final composition of matter has been described in a separate patent application. A description of the Method of Standardization of the product will be described in yet a different patent application.

This product may be administered orally to individual animals by either drenching or dosing with a solution of the product. It may be fed to individual animals by mixing it into the milk fed to that animal. It may be fed by some unique system as as has been described above for poultry. It may be mixed with a thickening agent and spread or sprayed on the udder and teats of sows. Or, it may be top-dressed on dry feed for swine and poultry. The feeding period should range from 3-7 days and should start on day 1 or 2 of life. Calves, foals, kids and lambs should be fed the product 5-7 days, starting on day 2 of life. Day one should be used for feeding colostrum. Chicks and turkey poults should be fed the product beginning on day one and continued for 2-3 days. Pigs should be fed the specialized product from day 2 for 4-7 days.

### Experimental Supporting Trials

Field trials with this mixture included with the freeze dried bacterial polysaccharide resulted in improved growth rate and weight gain over the use of the bacterial polysaccharide alone. Use of the specially collected rumen fluid bacterial polysaccharide resulted in less sick animals, less mortality and fewer treatments required in calves.

### Trials

### New Mexico Calf Treatment Trial

The objective of this study was to compare 3 different treatments for calves. The main exercise here was to find if freeze-drying was an acceptable treatment for the autoclaved rumen fluid. To ensure that each treatment was randomly assigned the treatment was assigned to the calves in the order they were delivered to the calf raiser. Both bull calves and heifer calves were treated. Each calf was assigned to the treatment group according to the order of delivery to the calf raising facility, the farm of origin and the sex of the calf. Bull calves derived from other farm(s) than C_Dairy were considered a separate subgroup. Each calf was assigned to the treatment group according to the color of the treatment that was next in the rotation. The rotation was determined to be white, green and red. There were 3 subgroups in the study: C_Dairy heifers, C_Dairy bulls and other dairies' bulls. The rotation of treatments was made within each of the subgroups. For example: Two heifers are delivered on Monday. The first is assigned to the white treatment, the second is assigned to the green treatment. The first bull delivered from C_Dairy is assigned to the White treatment. The first bull from other dairies is assigned to the White treatment. On Tuesday, four more heifers are delivered. The first is assigned to the red treatment, then white, green and red. The same type of rotation was used for C_Dairy bulls and other dairies' bulls. The C_Dairy bulls were separated from the other bulls for two reasons. First there were records available from C_Dairy on dam age and colostrum administration. Second, the other bull calves were assimilated from several other dairies and owned by the calf raisers instead of C_Dairy.

The calf raisers recorded the calf's dam's number (when available) and birth date (delivery date was considered acceptable). They also recorded which treatment the calf was assigned to. If available, they were asked to check the appropriate space if the calf was a twin or if the cow had to be helped to deliver the calf (the calf was pulled). The calf should be weighed on arrival. Colored grease markers were used to mark each pen to allow the workers the ability to quickly identify the treatment group the calves are assigned to.

The treatment assigned was given for seven days. The calves were treated only 1 time per day in the morning. The calf was to receive colostrum the first day and then receive the treatment for 7 days. The medicines used for each treatment group were:

Treatment—White Calf Treatment Group—White Powder Treatment—Freeze dried autoclaved rumen fluid with probiotics, chelated trace minerals, amino acids.

Positive control—Green Calf Treatment Group—Green Liquid Treatment—

Autoclaved liquid rumen fluid colored with cake coloring.

Negative Control—Red Calf Treatment Group—Red Powder Treatment—Milk powder colored with Kool-Aid®.

The mixing and feeding instructions given to the calf feeders were:

Mix the treatment in the milk prior to feeding the calf. The treatment may be mixed for several calves at once, however it may tend to settle out if allowed to stand. The bottles should be filled immediately after mixing the treatment and then inverted once or twice prior to feeding. If the milk has to stand in a five-gallon container following mixing prior to feeding or pouring into bottles, remix the container prior to pouring up for the calves. Once mixed the milk will have a color the same as the treatment group. Pink milk to the calves with a red marked pen, white milk (yellowish-gray color) to the calves with white marked pen and green milk to calves with a green marked pen.

The powder treatment is mixed at 2 level teaspoons (tsp—small spoon) per bottle. When mixing for several calves, mix % cup rounded plus two tablespoons level per 5 gallon bucket.

The liquid treatment is mixed at the rate of 8 cc per bottle or 80 cc per 5 gallon bucket. Shake well before drawing out this treatment. A needle is not needed to draw it out of the bottle. The tops have slits that will allow a syringe tip to be inserted to facilitate drawing out the treatment.

The monitoring instructions used during this trial are as follows:

Although the treatment is only given for seven days, the effects are expected to last until weaning. The calves should be monitored daily until weaning. At weaning the calves should be weighed and the weight recorded on the sheet containing the calfs birth date and dam #.

Should any of the animals become sick, treat them, as is your normal practice and record the date and the medicaments used.

Daily—Record any calves that are sick, and the medicines administered.

Results: The weight gains were better for the treated animals in two of the trial groups. The group of heifers did not show the same response. The difference in the incoming weight of the three treatment groups within the heifer group may have contributed to this lack of response. The difference in the gain between the treatment group and the average of the two control groups as shown below is 6.3 #, 6.2 # and 0.9# respectively. Due to irregularities in the recording of illnesses and differences in the treatments used between groups (C_Dairy vs Purchased) these data were not included into the analysis.

New Mexico Calf Trial

| Treat- | Number of Calves | In Weight | Out Weight | Gain During |
|---|---|---|---|---|
| ment | in Group | in Pounds | in Pounds | Trial |
| PBG | 17 | 88.4 | 150.6 | 61.2 |
| PBR | 15 | 93.9 | 157.9 | 64.0 |
| PBW | 17 | 90.5 | 159.4 | 68.9 |
| CBG | 17 | 90.7 | 156.8 | 66.1 |
| CBR | 17 | 89.2 | 155.4 | 66.2 |
| CBW | 17 | 88.8 | 161.2 | 72.4 |
| CHG | 17 | 77.8 | 135 | 57.2 |
| CHR | 16 | 74.8 | 134.5 | 59.7 |
| CHW | 17 | 82.0 | 141.3 | 59.3 |

P = PURCHASED
C = C_DAIRY
B = BULL CALF
H = HEIFER CALF
G = POSITIVE CONTROL with liquid product
R = NEGATIVE CONTROL
W = TREATMENT with freeze dried product

### Texas Calf Treatment Trial

The objective of this study was to compare 3 different treatments for calves. To ensure that each treatment was randomly assigned the treatment was assigned to the calves in the order they were born. Both bull calves and heifer calves were treated. Each calf was assigned to the treatment group according to the color of the card the calfs number appeared on. The cards were printed on three different color card stock. The assignment of the treatment used for each treatment group was:

Pink Calf card—Red Powder Treatment—Negative Control

White Calf card—White Powder Treatment—Warm Air Dried Positive Control

Green Calf card—Green Liquid Treatment—Treatment Group

The calf's dam's number and birth date were recorded on the cards. The workers were asked to check the appropriate space if the calf is a twin or if the cow needed assistance to deliver the calf (the calf was pulled).

The treatment assigned was given for seven days. The calves were treated only 1 time per day in the morning. The calf was to receive colostrum the first day and treatment for the next 7 consecutive days. The calf feeder was asked to circle the day of birth and then X each day the treatment is given.

The mixing and feeding instructions given to the calf feeders were:

Mix the treatment in the milk prior to feeding the calf. The treatment may be mixed for several calves at once, however it may tend to settle out if allowed to stand. The bottles should be filled immediately after mixing the treatment and then inverted once or twice prior to feeding. If the milk has to stand in a five-gallon container following mixing prior to feeding or pouring into bottles, remix the container prior to pouring up for the calves. Once mixed the milk will have a color the same as the card. Pink milk to the calves with a pink card, white milk (grayish color) to the calves with white cards and green milk to calves with a green card.

The two powder treatment are mixed as 2 level teaspoons (tsp—small spoon) per bottle. When mixing for several calves, mix 6 Tablespoons (tbsp—large spoon) per 5 gallon bucket

The liquid treatment is mixed at the rate of 8 cc per bottle or 80 cc per 5-gallon bucket. Shake well before drawing out this medicine.

Although the treatment is only given for seven days, the effects are expected to last until weaning. The calves should be monitored daily until weaning or until the individual pages are collected (this may be done prior to weaning if the calves appear normal).

The monitoring instructions used during this trial are as follows:

Daily—Record the score of the manure from the calf. The scores to be used are:
1. Normal (1)—Firm but not hard. Original form is distorted slightly after dropping to floor and settling.
2. Soft (2)—Does not hold form, piles but spreads slightly. Similar to soft serve ice cream.
3. Runny (3)—Spreads readily to about ¼ of an inch (6 mm) in depth. Similar to pancake batter.
4. Watery (4)—Liquid consistency, splatters. Similar to orange juice.

If there is some question as to whether the manure is one score or another, for example: soft or runny, just list both scores for that day. If diarrhea develops during the day, simply write in the second score with PM after it for the later observation. If diarrhea continues for 4 days and it is watery for the four days this should be recorded each day as 4. An example of the records follows. In the example the first day (November 1) was normal and this is recorded as a 1. The second day (November 2) the calf had soft manure in the morning and watery diarrhea in the afternoon. This would be recorded as a 2 for the soft manure in the morning and as a 4 followed by PM for the watery manure in the afternoon. The next three days the calf has watery diarrhea (Nov. 3-5, and recorded as a 4). The calf is better on November 6 and the manure is not runny but really isn't firm enough to be soft. This would be recorded as a 2 for soft and a 3 for runny. On November 7 the calf is headed for recovery and the manure is soft recorded as a 2.

| Nov 1 | Nov 2 | Nov 3 | Nov 4 | Nov 5 | Nov 6 | Nov 7 |
|---|---|---|---|---|---|---|
| 1 | 2 | 4 | 4 | 4 | 2-3 | 2 |
| | 4PM | | | | | |

Treatment descriptions are:

Green Liquid Treatment—Autoclaved liquid rumen fluid

Red Powder Treatment—Milk powder with red Kool-Aid®

White Powder Treatment—Warm Air Dried Rumen Fluid on ground rice base with added probiotics, vitamins and trace minerals.

### Results:

There were no differences in manure consistency scores between treatments. The number of antibiotic treatments administered to animals for diarrhea was reduced by 50% for treated calves. There were no deaths of treated calves but 4 and 3 deaths in the two control groups. No body weights were recorded in this trial.

Texas Calf Trial

| | # calves | # antibiotic | | # animals |
|---|---|---|---|---|
| Treat- | per | treatments | # | treated with |
| ment | treatment | for group | Deaths | antibiotics |
| GH | 12 | 9 | 0 | 6 |
| RH | 13 | 12 | 3 | 5 |
| WH | 13 | 14 | 2 | 7 |
| GB | 14 | 1 | 0 | 1 |
| RB | 13 | 8 | 1 | 5 |
| WB | 11 | 9 | 1 | 6 |
| Total G | 26 | 10 | 0 | 7 |
| Total R | 26 | 20 | 4 | 10 |
| Total W | 24 | 23 | 3 | 13 |

G = Liquid product treatment
R = Negative control
W = Heat dried positive control
B = Bull calf
H = Heifer calf
WB - Two of these calves died within 24 hours following birth

I claim:

1. A method for the promotion of growth and weight gain, the abatement of diarrheal disease and the reduction in mortality in a farm animal comprising the administration of bacterial polysaccharides derived from dried rumen fluid combined with nutritional aids for the first 1-7 days of life.

2. A method according to claim 1, in which the farm animal is a calf.

3. A method according to claim 1, in which the farm animal is a pig.

4. A method according to claim 1, in which the farm animal is a chick.

5. A method according to claim 1, in which the farm animal is a turkey poult.

6. A method according to claim 1, in which the farm animal is a foal.

7. A method according to claim 1, in which the farm animal is a kid.

8. A method according to claim 1, in which the farm animal is a lamb.

9. A method according to claim 1, in which the nutritional aids contain 20-95% of the amino acid threonine.

10. A method according to claim 1, in which the nutritional aids contain 5-70% of a monosaccharide.

11. A method according to claim 1, in which the nutritional aids contain the recommended daily dose of vitamins A, D, E for the newborn of the species being fed.

12. A method according to claim 1, in which the nutritional aids contain a specially selected probiotic for the species being fed.

13. A method according to claim 1, in which the nutritional aids contain the recommended daily dose of Thiamine, Riboflavin, Pyridoxine, Pantothenic acid, Niacin, Biotin, Folic acid, B₁₂ and Choline for the newborn of the species being fed.

14. A method according to claim 1, in which the nutritional aids contain the organic trace minerals Manganese, Zinc, Copper, and Selenium.
